(19) United States
(12) Patent Application Publication
(10) Pub. No.: US 2022/0002572 A1
(43) Pub. Date: Jan. 6, 2022

(54) HYDROPHILIC COATING COMPOSITION FOR DUAL COATING AND HYDROPHILIC COATING METHOD USING SAME

(71) Applicant: Noanix Corporation, Cheongju-si (KR)

(72) Inventor: Hyung Joon CHOI, Cheongju-si (KR)

(21) Appl. No.: 17/090,693

(22) Filed: Nov. 5, 2020

(30) Foreign Application Priority Data
Jul. 1, 2020 (KR) 10-2020-0080983

(51) Int. Cl. C09D 105/08; C09D 175/08; C08K 5/21; C09D 7/63; B05D 7/00

(52) U.S. Cl. CPC C09D 105/08; C09D 175/08; C08K 5/21; B05D 2518/00; B05D 7/544; B05D 2503/00; C09D 7/63

(57) ABSTRACT

A hydrophilic coating composition and a hydrophilic coating method using the same are disclosed. The hydrophilic coating composition can include a first coating solution, a second coating solution, and a crosslinking agent. The first coating solution includes a polyurethane-based compound, and the second coating solution includes a polysaccharide. The coating composition can provide a coating that is strongly hydrophilic, highly biocompatible, thin, and flexible. Thus, when the coating composition is applied to an invasive medical device, lubricity can be greatly increased, thereby preventing damage to the human body, such as injury, tissue abrasion and the like.

### CROSS REFERENCE TO RELATED APPLICATION

The present application claims priority based on Korean Patent Application No. 10-2020-0080983, filed on Jul. 1, 2020, the entire content of which is incorporated herein for all purposes by this reference.

### BACKGROUND OF THE DISCLOSURE

### 1. Technical Field

The present disclosure relates to a hydrophilic coating composition and a hydrophilic coating method using the same.

### 2. Description of the Related Technology

Recently, the development of medical devices that complement surgical procedures is being actively conducted, and examples thereof include various vascular catheters that are used to treat the circulatory system in, for example, aortic surgery, etc., stents that strengthen arterial walls and prevent occlusion after angioplasty, and the like. Additional examples of such devices include heart valves, artificial pacemakers and orthopedic implants.

The devices described above are often made of plastics and metals that are present for a long time in the human body. These typically have surface-area characteristics considerably different from those of human organs, which are hydrophilic, slippery and biocompatible. These non-biocompatible invasive devices are regarded as foreign objects by the body's defense system, often causing inflammation and thrombosis.

Korean Patent No. 10-0669629 discloses the related technology.

The disclosure of this section is to provide background information relating to the invention. Applicant does not admit that any information contained in this section constitutes prior art.

### SUMMARY OF THE DISCLOSURE

In the case of medical devices that is to be inserted and moved through body tissue, lubricity is also important. Most metals and plastics have poor lubricity with regard to body tissue, so mechanical friction may occur when the device passes through the tissue. The surface of the devices designed and manufactured using such materials may need to be made hydrophilic, slippery and biocompatible through appropriate coating.

In order to address the foregoing, the inventors of the present disclosure devised a hydrophilic coating solution having lubricity, abrasion resistance and biocompatibility. The coating solution is suitable for coating vascular catheters, guide wires and other medical devices, and is applicable to a wide range of polymer and metal substrates.

Accordingly, an aspect of the present disclosure is to provide a hydrophilic coating composition including a first coating solution including a polyurethane-based compound, a second coating solution including a polysaccharide, and a crosslinking agent.

Another aspect of the present disclosure is to provide a hydrophilic coating method using the hydrophilic coating composition described above.

The present disclosure provides a hydrophilic coating composition, including: a first coating solution including a polyurethane-based compound; a second coating solution including a polysaccharide; and a crosslinking agent.

In an embodiment of the present disclosure, the first coating solution may be bound to a substrate, and the second coating solution may be bound to the first coating solution, which is bound to the substrate, by the crosslinking agent.

In an embodiment of the present disclosure, the composition may be biocompatible.

In an embodiment of the present disclosure, the polyurethane-based compound may be polyether polyurethane.

In an embodiment of the present disclosure, the polysaccharide may be hyaluronic acid.

In an embodiment of the present disclosure, the crosslinking agent may be a polyaziridine-based or polyisocyanate-based crosslinking agent.

In an embodiment of the present disclosure, the second coating solution may further include polyether polyurethane.

In an embodiment of the present disclosure, the weight ratio of the polysaccharide to the polyether polyurethane may be 10:0.5-2.

In addition, the present disclosure provides a hydrophilic coating method, including: a) coating the surface of a substrate with a first coating solution; b) drying the substrate coated in step a); c) coating the substrate dried in step b) with a second coating solution; and d) drying the second coating solution applied in step c).

In addition, the present disclosure provides a substrate subjected to hydrophilic coating using the method described above.

The coating solution of the present disclosure is capable of providing a coating that is strongly hydrophilic, highly biocompatible, thin, and flexible. Therefore, when the coating solution of the present disclosure is applied to an invasive medical device, lubricity can be greatly increased, thus preventing damage to the human body, such as injury, tissue abrasion and the like.

### BRIEF DESCRIPTION OF THE DRAWINGS

FIG. 1A and FIG. 1B show the results of comparison of the frictional force depending on the presence or absence of an additive in a second coating solution.

FIG. 2A and FIG. 2B show the results of comparison of the frictional force before and after coating using a hydrophilic coating solution of the present disclosure.

### DETAILED DESCRIPTION OF EMBODIMENTS

An aspect of the present disclosure pertains to a hydrophilic coating composition including a first coating solution including a polyurethane-based compound, a second coating solution including a polysaccharide, and a crosslinking agent.

In the present disclosure, polyurethane is a generic term for a polymer compound formed using a urethane linkage, achieved through bonding between an alcohol group and an isocyanate group. In the present disclosure, the polyurethane-based compound is not particularly limited, so long as it exhibits the properties of polyurethane, and there may be provided various polyurethane-based compounds using a compound having an alcohol group or an isocyanate group within a range of modification obvious to those skilled in the art.

In the present disclosure, the polysaccharide is a polymeric carbohydrate molecule composed of a long chain of monosaccharide units joined by glycoside bonds.

As used herein, the term “hydrophilic” means that droplets do not easily form beads on the surface of a hydrophilic material but droplets have a contact angle of less than 45° therewith and tend to spread easily on the surface thereof.

The hydrophilic coating composition of the present disclosure may include one or more additives typically used in coating formulations, for example, surfactants, preservatives, viscosity modifiers, pigments, dyes and other additives known to those skilled in the art.

In the present disclosure, the first coating solution may be bound to a substrate, and the second coating solution may be bound to the first coating solution, which is bound to the substrate, by the crosslinking agent.

The hydrophilic second coating solution in the coating solution of the present disclosure imparts lubricity to a coated medical device when brought into contact with an aqueous medium. The first coating solution is interposed as the middle layer between the hydrophilic second coating solution and the surface of the medical device, and has superior adhesion to the surface of the medical device.

The crosslinking-agent compound is used to bind the second coating solution polymer to the first coating solution polymer. Therefore, the hydrophilic polymer in the second coating solution is chemically attached to the layer of the first coating solution. The cured first coating solution absorbs a very small amount of water, so the coated medical device is capable of maintaining adhesion when brought into contact with an aqueous medium. Simultaneously, the second coating fixed to the first coating may confer lubricity.

In the present disclosure, the crosslinking agent may be applied without limitation, so long as it is a compound capable of binding a layer coated with the first coating solution (hereinafter, referred to as a “first coating layer”) and a layer coated with the second coating solution (hereinafter, referred to as a “second coating layer”) to each other. In embodiments, the crosslinking agent is an aziridine-based crosslinking agent or an isocyanate-based crosslinking agent, but is not limited thereto. In addition, the crosslinking agent may be used by being added to the first coating solution, or may be used separately from the first coating solution.

In the present disclosure, the substrate is an object that is coated with the hydrophilic coating solution, and there is no particular limitation thereon. In embodiments, the substrate is an invasive medical device. Typical examples of such a medical device may include, but are not limited to, a catheter, a balloon catheter, a guide wire, an endotracheal tube, an implant, and the like.

In the present disclosure, the composition may be biocompatible.

Here, the term “biocompatible” refers to a property that does not cause any harm or side effects to a living body when a material is administered or applied to the living body.

In the present disclosure, the polyurethane-based compound may be polyether polyurethane, but is not limited thereto.

In the present disclosure, the polysaccharide may be hyaluronic acid, but is not limited thereto.

In the present disclosure, the crosslinking agent may be a polyaziridine-based or polyisocyanate-based crosslinking agent, but is not limited thereto.

In the present disclosure, the second coating solution may further include polyether polyurethane, and the polyether polyurethane included in the second coating solution may be the same as the polyether polyurethane included in the first coating solution, or may be another polyether-polyurethane series compound.

In the present disclosure, the weight ratio of the polysaccharide to the polyether polyurethane may be 10:0.5-2, in one embodiment, 10:0.5-1.5, and in another embodiment, 10:1. When the substrate is coated with the coating solution at the above mixing ratio, a coating having low frictional force and high durability may be provided.

Another aspect of the present disclosure pertains to a hydrophilic coating method including a) coating the surface of a substrate with a first coating solution, b) drying the substrate coated in step a), c) coating the substrate dried in step b) with a second coating solution, and d) drying the second coating solution applied in step c).

The coating may be formed through a coating process that is typical in the art using the hydrophilic coating solution of the present disclosure. In embodiments, a dip-coating process is performed, but the present disclosure is not limited thereto.

Still another aspect of the present disclosure pertains to a substrate subjected to hydrophilic coating using the above method. Since the coating solution of the present disclosure provides a coating that is strongly hydrophilic, highly biocompatible, thin, and flexible, the coated substrate is greatly increased in lubricity, thus preventing damage to the human body, such as injury, tissue abrasion and the like.

A better understanding of the present disclosure will be given through the following examples, which are merely set forth to illustrate the present disclosure and are not to be construed as limiting the scope of the present disclosure.

### EXAMPLE 1

### Preparation of Coating Solution

### Preparation of First Coating Solution

10 g of 1085 A 15 resin, which is a polyether-polyurethane-based compound, was quantified and added with 1 L of a solvent composed of ethanol and water at a ratio of 9:1. Then, stirring was performed at 600 to 800 rpm using a magnetic bar so that the resin was completely dissolved. Then, a polyaziridine-based crosslinking agent, HD-100, was quantitatively added (3 g per 1 L) using a dropper, and the solution to which HD-100 was added was stirred (400-600 rpm) at room temperature for 5 min using a magnetic bar, thereby preparing a first coating solution containing the crosslinking agent.

### Preparation of Second Coating Solution

50 g of a hyaluronic acid powder was quantified and placed in a 1 L bottle, 500 ml of ethanol (EtOH) was placed in the bottle, and the hyaluronic acid powder was thoroughly dispersed by stirring. Then, 500 ml of distilled water (DW) was slowly added thereto and stirred until the hyaluronic acid powder was dissolved. Complete dissolution of the hyaluronic acid powder was confirmed, after which stirring was conducted until bubbles disappeared, and 5 g of 5604A, which is a polyether-polyurethane-based compound, was added and stirred until dissolved, thereby preparing a second coating solution.

### EXAMPLE 2

### Coating of Substrate Using Hydrophilic Coating Solution

A coating object was washed with a mixed solution of isopropyl alcohol (IPA) and DW and then dried. Then, the coating object was immersed in the first coating solution prepared in Example 1, coated, and dried in an oven (60° C., 10 min). Then, the object was cooled at room temperature for about 5 to 10 min, immersed in the second coating solution prepared in Example 1, coated, and dried in an oven (60° C., 120 min). Thereafter, the coated object was completely immersed in distilled water for washing, allowed to stand for 20 min, further washed with distilled water, and then dried.

### EXAMPLE 3

### Observation of Increased Lubricity of Second Coating Solution Containing Additive

In order to confirm the improved effect of the coating solution when polyether polyurethane is included as an additive in the second coating solution, a frictional force test was conducted.

Specifically, the upper portion of a catheter sample coated through the method of Example 2 was fixed using a clamp above a water tank in an apparatus, after which the sample was pulled at a predetermined speed ten times while the frictional force was measured using a frictional force sensor to determine surface smoothness.

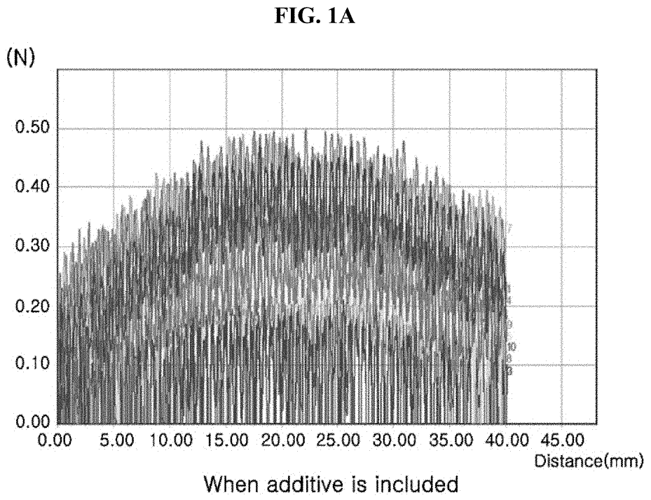

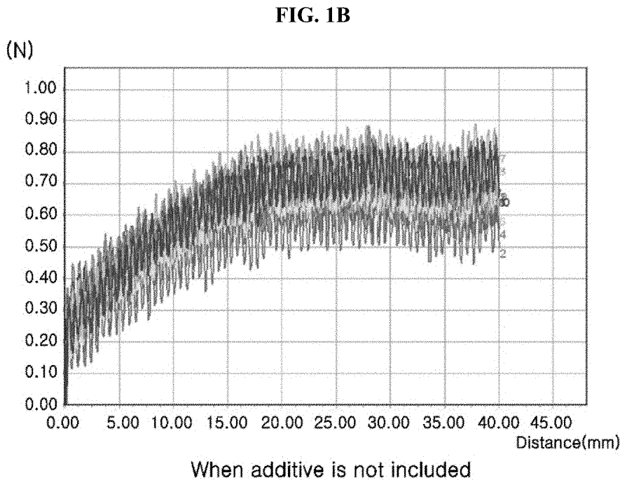

As shown in FIG. 1A and FIG. 1B, the frictional force of the sample containing the additive was measured to be significantly low. Thereby, it can be confirmed that, when polyether polyurethane was included as the additive, the surface smoothness of the substrate was remarkably increased through a synergistic effect with hyaluronic acid.

### EXAMPLE 4

### Comparison of Frictional Force of Substrate Before and After Coating

In order to confirm the effect of the surface coating, the surface of the sample before and after coating was subjected to a frictional force test in the same manner as in Example 3.

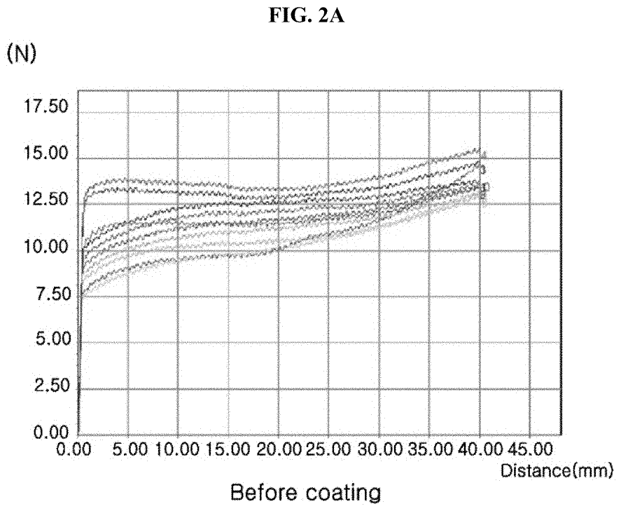

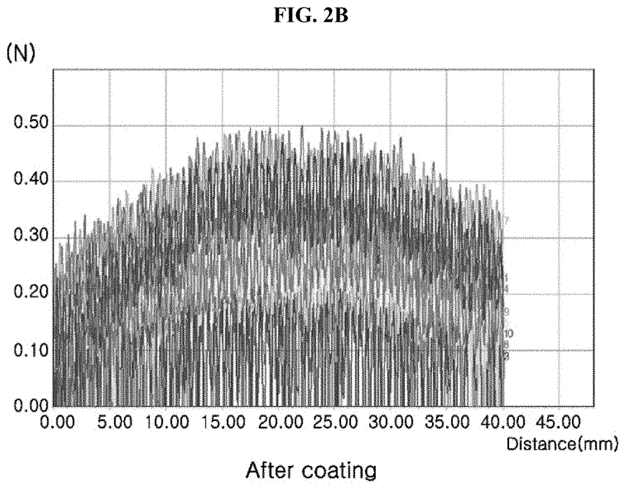

As shown in FIG. 2A and FIG. 2B, it can be confirmed that the surface frictional force after coating treatment exhibited a remarkably low value compared to the surface frictional force before coating treatment, which means that the surface smoothness of the substrate after coating was remarkably increased.

Although embodiments of the present disclosure have been disclosed for illustrative purposes, those skilled in the art will appreciate that various modifications, additions and substitutions are possible, without departing from the scope and spirit of the present disclosure as disclosed in the accompanying claims.

What is claimed is:

1. A hydrophilic coating composition, comprising:
a first coating solution comprising a polyurethane-based compound;
a second coating solution comprising a polysaccharide; and
a crosslinking agent configured to link the first and second coating solutions.

2. The hydrophilic coating composition of claim 1, wherein the crosslinking agent is configured to bind the first coating solution to a substrate, and bind the second coating solution to the first coating solution.

3. The hydrophilic coating composition of claim 1, wherein the composition is biocompatible.

4. The hydrophilic coating composition of claim 1, wherein the polyurethane-based compound comprises polyether polyurethane.

5. The hydrophilic coating composition of claim 1, wherein the polysaccharide comprises hyaluronic acid.

6. The hydrophilic coating composition of claim 1, wherein the crosslinking agent comprises a polyaziridine-based crosslinking agent or a polyisocyanate-based crosslinking agent.

7. The hydrophilic coating composition of claim 1, wherein the second coating solution further comprises polyether polyurethane.

8. The hydrophilic coating composition of claim 7, wherein a weight ratio of the polysaccharide to the polyether polyurethane is about 10:0.5-2.

9. A hydrophilic coating method, comprising:
first coating a surface of a substrate with a first coating solution comprising a polyurethane-based compound;
drying the coated substrate;
second coating the dried substrate with a second coating solution comprising a polysaccharide, using a crosslinking agent; and
drying the second coating solution applied in the second coating.

10. A substrate comprising:
a first surface coated with a first coating solution comprising a polyurethane-based compound, the first surface further coated with a second coating solution comprising a polysaccharide.
